(19) United States
(12) Patent Application Publication
(10) Pub. No.: US 2021/0208681 A1
(43) Pub. Date: Jul. 8, 2021

(54) SYSTEMS AND METHODS FOR TELEPATHIC IMPLANT AND ACCELERATED HUMAN COMPUTER INTERACTION

(71) Applicant: EYETHENTICATE, LLC, Boca Raton, FL (US)

(72) Inventor: Austin H. Lubetkin, Boca Raton, FL (US)

(21) Appl. No.: 17/141,127

(22) Filed: Jan. 4, 2021

Related U.S. Application Data
(60) Provisional application No. 62/957,121, filed on Jan. 4, 2020.

(51) Int. Cl. G06F 3/01; A61B 5/386; A61B 5/00

(52) U.S. Cl. CPC G06F 3/015; A61B 5/747; A61B 5/7465; A61B 5/386

(57) ABSTRACT

The present invention provides a non-physical communication interface between humans and machines which allows for specific commands with a high degree of success. Systems and methods provided for herein allow for a non-physical, non-verbal and non-audible communication interface between humans and machines, or a telepathic communication from a human to a computing device. Embodiments of the invention allow for human telepathic control of machines, or allow for human to human telepathic communication through the use of one or more computers or machines.

### CROSS-REFERENCE TO RELATED APPLICATIONS

This application claims the benefit of U.S. Provisional Application No. 62/957,121, having a filing date of Jan. 4, 2020, the disclosure of which is hereby incorporated by reference in its entirety and all commonly owned.

### FIELD OF INVENTION

The present invention relates to systems and methods for telepathic implant and accelerated human computer interaction. More particularly, the present relates to systems and methods for human telepathic communication with computing devices.

### BACKGROUND

Technology has become an increasing integral part of modern society. Many individuals with varying ability have encountered significant challenges when interfacing with technology. These challenges can include limits on physical mobility or neurological processing of writing. In addition, many technological solutions that create an accessible interface have both a high technical barrier to entry (processing power, hardware complexity) and a high financial barrier to entry (proprietary software, limited commercial availability). In addition, activities that require maneuvers in high gravity environments come with physical limitations due to the intense physical force exerted by gravity, especially in critical or failing systems. Thus there remains an unmet need to improve human and machine interaction which requires little human physical action.

The human conscious is the result of electric impulses in the brain. These impulses come in many forms and are collectively referred to as brain waves. It is relatively known that the frequency and amplitude of the brain waves results in the classification and naming of the waves observed in the brain. Brainwaves are commonly detected through the use of electroencephalogram (EEG) machines. These involve placing small conductive metal disks on the surface of the head which detect electrical activity in the brain. The data resulting from an EEG is similar to brainwaves in the sense that it is an electrical frequency with qualities like amplitude and peaks/valleys. EEG devices are highly commercially available, safe, and are commonly used by medical professionals. A notable use of EEG is to detect activity in the brain relevant to a seizure.

Some attempts have been made to providing human EEG interface with devices, but to date, none have been widely adopted for varying reasons, but ultimately because of the complexity, or failure to establish reliable communication. A common implementation is to stare at a target and see measures of brain activity increase as focus is directed at a visible target. This data can clearly and objectively detect extreme binary variants between focused or not focused but struggles to detect and perform at a significant level with more subtle and intentional variants like the difference between aiming for 25% activity or 50% activity. Ultimately the techniques adopted make it extremely difficult to perform a specific command because intentional selection added a layer of complexity that made input inaccurate. Others have attempted to increase the precision of detection by redesigning the technology to provide an invasive surgically implanted interface to increase the precision of detection. This approach assumes language as something that can be directly correlated to brainwaves with the intent of using AI and machine learning to recognize thought words or phrases. Thus there remains an unmet need to provide a system or method which can accurately provide a non-physical communication interface between humans and machines which allows for specific commands with a high degree of success.

### SUMMARY OF INVENTION

The present invention provides a non-physical communication interface between humans and machines which allows for specific commands with a high degree of success.

In some aspects of the invention systems and methods are provided which provide for a non-physical, non-verbal and non-audible communication interface between humans and machines.

Some aspects of the present invention provide systems and methods to provide telepathic communication human to a computing device.

Further aspects of the present invention provide for interfaces allowing for two-way communication between a human and a machine, and/or three-way communication allowing for the non-physical, non-verbal, and non-audible (or telepathic) communication between humans by and through one or more machines or electronic devices.

### BRIEF DESCRIPTION OF THE DRAWINGS

Examples illustrative of embodiments of the disclosure are described below with reference to figures attached hereto. In the figures, identical structures, elements or parts that appear in more than one figure are generally labeled with the same numeral in all the figures in which they appear. Dimensions of components and features shown in the figures are generally chosen for convenience and clarity of presentation and are not necessarily shown to scale. Many of the figures presented are in the form of schematic illustrations and, as such, certain elements may be drawn greatly simplified or not-to-scale, for illustrative clarity. The figures are not intended to be production drawings. The figures (Figs.) are listed below.

FIG. 1 illustrates at least one exemplary embodiment of a telepathic communication system.

FIG. 2 illustrates at least one exemplary embodiment of a telepathic communication method.

FIG. 3 illustrates at least one exemplary embodiment of a crash mitigation and avoidance device utilizing the inventive system.

FIG. 4 illustrates at least one exemplary embodiment of a journalizing/data recording device utilizing the inventive system.

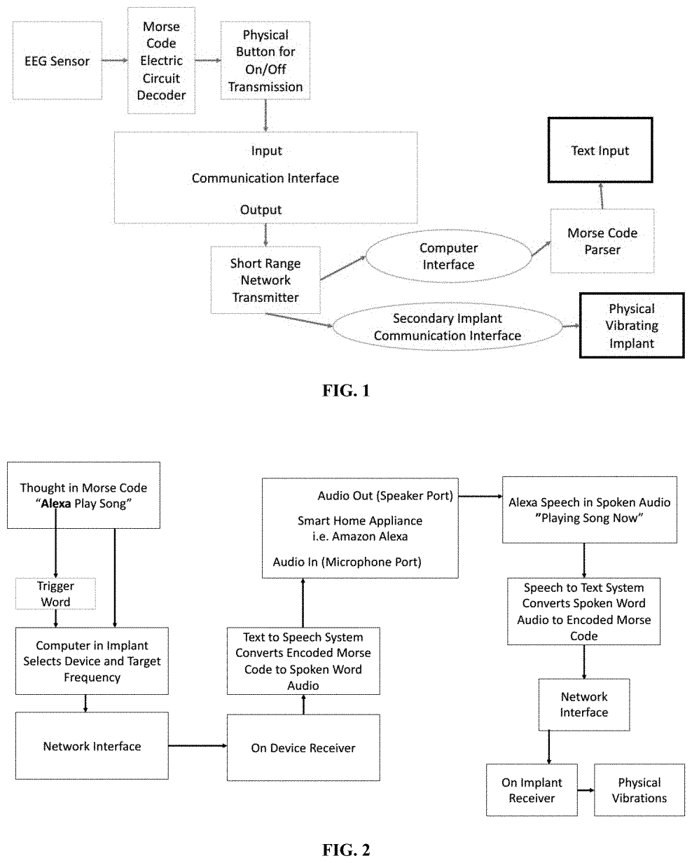

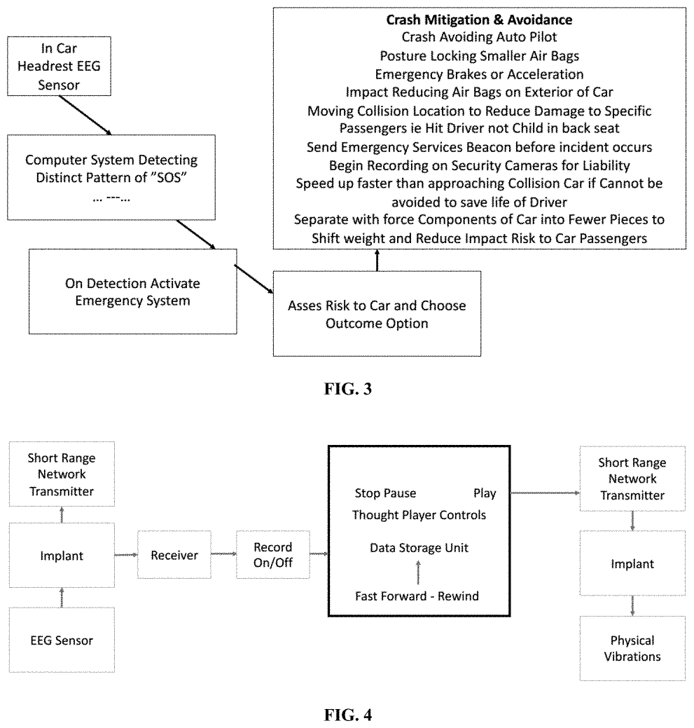

It should be clear that the description of the embodiments and attached Figures set forth in this specification serves only for a better understanding of the invention, without limiting its scope. It should also be clear that a person skilled in the art, after reading the present specification could make adjustments or amendments to the attached Figures and above described embodiments that would still be covered by the present invention.

### DETAILED DESCRIPTION

The present invention is directed to methods, systems and devices for telepathic communication from human to a computing device. The present invention accomplishes its success by focusing on extreme inputs to deliver accuracy and precision commands. Generally the methods, systems and devices rely on restructuring language away from a full alphabet to a binary input by communicating in Morse code.

Implant is a combination of embedded EEG sensors, quartz vibrating implant, short range transmitter, short range receiver, implanted button to turn transmitters on and off.

Peaks and valleys of brainwaves are identified as Morse code. This is then transmitted in the short range. This can either be picked up by a computer terminal or another implant.

If the signals are picked up by another implant then the quartz chip vibrates with Morse code and messages are felt nonverbally. This is a form of short range telepathic communication that requires no visible or audible stimuli.

If picked up a computer then these messages can be used to type and control a computer with Morse code. Eye movements can control a mouse pointer and blinks can be clicks with a single eye blink being a right click. This is completely hands-free computer interface that can be used by anyone even someone who is completely paralyzed.

Once a user regularly uses the software, their thoughts will start to be in the format of Morse code. Speakers of other formats of language like sign language describe thinking in sign language. This phenomenon presents a unique opportunity when a user's familiarity turns thoughts to Morse code. These binary formatted thoughts can be detected and recorded in addition to conscious and intentional thoughts.

Using machine learning, it becomes possible to see how a person thinks through a problem or task that is seen on a computer screen. This can then be used to predict how the task would be completed. This can start small with auto filling words but can advance as familiarity with a thought process is further understood. Machine learning can slowly branch back the steps of the thought patterns and eventually it will become possible to go from perception to instant task completion. This would mean a user would see something on the screen, process it, and an AI would complete the task before they could consciously do it. This accelerates all human computer interactions and would allow any task that would normally be completed on a computer to be completed at an accelerated rate.

This unique type of AI that can do anything is the first of what could be considered a Strong AI. It's a generalist that can complete any task which is one requirement. It can predict and think through conscious thoughts the same as the human it is trained on. This would mean it could complete a Turing test by going from prompt to conscious human response. One unique feature that differs from standard perceptions of Strong AI that is also a feature of weak AI is that its capped at human intelligence. It can't go beyond the brain capacity of the thought patterns its trained on. Weak AI can complete a single task really well and develops complex logic to complete a specific task. This strong AI doesn't develop its own logic and instead learns to make its own logic by the process and actions of a real human brain.

Another potential use is for security purposes, a user can think out an SOS and it would initiate emergency features. This could become a continuous security feature with the implant. If someone falls in their home and can't get up they could think an SOS and then beacons could rely this message to emergency services. If someone is afraid they are going to get mugged, they could think an SOS and their phone could call 911. If someone is flying a plane and loses control, they could think an SOS and initiate emergency procedures hands-free and instantly. This process of thinking an SOS could go from incident to feature faster than physically seeking help. If someone is falling they could think an SOS and then an airbag could prevent damage to their hip in a fall before they even touch the ground.

The following detailed description is merely exemplary in nature and is in no way intended to limit the scope of the invention, its application, or uses, which may vary. The invention is described with relation to the non-limiting definitions and terminology included herein. These definitions and terminology are not designed to function as a limitation on the scope or practice of the invention, but are presented for illustrative and descriptive purposes only.

As used herein “human interface device” means one or more devices which read, or assist in reading, communicating or transmitting EEG signals from a human mental input. In some embodiments, the human interface device may monitor one or more other muscle groups, including, without limit, facial muscles, to refine EEG inputs.

As used herein “communicating . . . in Morse code” means that a user could either speak the dots with the internal voice in their head creating spikes of activity or could otherwise focus on and off a target to create the brain activity for the Morse code. Because, the data is simplified it becomes easier to parse the high amount of variation typically seen in EEG data.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

### Method

The present invention provides for a method for telepathically communicating with a computing device, which includes obtaining one or more human interface device containing at least one EEG sensor, communicating one or more words, letters, numbers, symbols, phrases or commands in Morse Code through thought, transmitting the EEG signal generated by the EEG in the human interface device to a computing device, and decoding the EEG signal into one or more words, letters, numbers, symbols, phrases or commands and converting said one or more words, letters, numbers, symbols, phrases or commands into verbal, visual, or vibrational communications.

Exemplary human interface devices are known in the art. It is intended that a human interface device be capable of receiving a human EEG. In at least one embodiment, the human interface device monitors additional human inputs such as facial muscles, assist in the proper interpretation of a human input. Certain embodiments of the human interface device include one or more signal transmitters for transmitting the received EEG signal from the human interface device to the computing device. In at least one embodiment, the human interface device further includes an amplifier which amplifies the EEG input. In at least one embodiment, the human interface device is an implantable device. In at least one embodiment, the human interface device is a wearable technology, such as a hat, headphones, or the like.

Once the human induced input signal is transmitted, it is received by a computing device which contains software or hardware for receiving the signal and decoding the received signal to a command to be executed by the computing device. It is appreciated that communication, in the sense of the present invention, is done by using extreme and exact inputs. Morse code, while discussed, is not the only form of extreme or absolute communication known in the art, and is intended to be at least one means of extreme communication. The communication is decoded by the decoder and the letters, words, phrases or commands that were communicated are executed by the computing device.

In totality, the method provides for an improved telepathic communication from a human to a computing device, thus allowing specific commands being employed with high probabilities of success.

### System

A system for enabling the human telepathic interaction with a computing device is provided. It should be appreciated that the systems described herein implement the one or more embodiments described above. In at least one embodiment the system includes a human interface device having at least one or more EEG sensors and one or more communication interface, and a computer interface device having one or more communication interface in communication with the communication interface of the human interface device and a Morse Code decoder.

The communication interface is intended to be used for communicating the received EEG signal to one or more devices, such as, without limiting the invention, a computing device. This communication may be accomplished wirelessly or with wire. Exemplary wireless communications include RF, Wi-Fi, Bluetooth, and radio transmission, among other methods of wireless communications known in the art.

As discussed in the methods, the received communications, (i.e. Morse code) is translated in to one or more words, letters, numbers, symbols, phrases or commands by the computer interface device. In embodiments of the invention, these received communications, after decoding, may be later communicated visually, audibly, or electronically.

It is appreciated that not only telepathically communicating with machines is desirable, but situations may arise that require human to human telepathic communication. Thus. in at least one embodiment, the system may further include one or more communication interface for communicating with a second human interface device.

### Devices

It should be appreciated that the systems and methods may be employed in specific devices to allow for a particular use.

In at least one embodiment, the system is implemented in a headset which includes, at least, a human interface device having at least one or more EEG sensors and one or more communication interfaces for communicating the received EEG signal to one or more locations. The use of the headset allows for a non-invasive and non-surgical means to provide a human with telepathic communication to an electronic device.

In at least one embodiment, the system is implemented in an emergency response actuating system which allows for the telepathic communication by a user to actuate directly, or through the use of a computing device, one or more emergency devices. Without intending to limit the invention, some examples of emergency response systems include an audible alarm, a silent alarm, an airbag, an auto-braking mechanism, an auto-pilot, an evasive maneuver protocol, parachute deployment, or ejection seat initiation.

Other devices may be employed for journaling or documenting thoughts which employ the system and methods coupled with one or more storage medium for receiving and storing the received EEG signal, the decoded message, or combinations thereof.

It is further appreciated that it may be desirable to communicate with certain legacy devices which natively do not have telepathic communications. In such embodiments, an adapter for converting the received EEG signal or Morse Code to one or more communication methods required for use of said electronic device. For example, a device that only operates as a result of a mechanical switch may have a mechanical input device to actuate the mechanical switch upon receipt of commands. As another non-limiting example, a device that only accepts voice commands, an adapter for receiving the EEG signal from the human interface device is used to translate the received signal and audibly communicate the signal through a voice generator and speaker.

In some instances it may be desirable for a human to receive a communication from an electronic device. In at least one non-limiting example, a fire alarm or a water leak sensor may want to communicate telepathically with a user to inform them of an event or alarming condition. In such embodiments, an input device for interfacing with the electronic device is used to receive the communication, encode it to the desirable signal (i.e. Morse code) and communicate the encoded command to a human interface device.

In at least one embodiment, systems may be incorporated to provide accident mitigation options. It should be appreciated that such devices are intended to be used in more extreme situations when the response to action time is shrunk to the point possible with this device. In at least one embodiment, the human interface device and/or EEG sensor is located in headrest of vehicle, or helmet of a pilot, skydiver or motorcyclist. Accident mitigation can include, for example, in a situation when a crash is inevitable moving the vehicle just enough that the impact of the crash would more so affect the driver than a child in the backseat. In some embodiments, when implemented in a motor vehicle, smaller airbags can be deployed as well as pre-locking the seatbelt to lock body positions and reduce things like whiplash. In at least one embodiment, accident mitigation may involve communications from the human interface device which mechanically weaken parts of a vehicle's body to crumple more effectively and reduce impact

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

1. A method for telepathically communicating with a computing device, the method comprising:
obtaining one or more human interface device containing at least one EEG sensor;
communicating one or more words, letters, numbers, symbols, phrases or commands in Morse Code through thought;
transmitting the EEG signal generated by the EEG in the human interface device to a computing device; and
decoding the EEG signal into one or more words, letters, numbers, symbols, phrases or commands and converting said one or more words, letters, numbers, symbols, phrases or commands into verbal, visual, or vibrational communications.

2. A system for enabling human telepathic interaction with a computing device, the system comprising:
a human interface device comprising:
one or more EEG sensors;
one or more communication interface;
a computer interface device comprising:
one or more communication interface in communication with the communication interface of the human interface device;
a Morse Code decoder.

3. The system of claim 2 further comprising translating the Morse code in to one or more words, letters, numbers, symbols, phrases or commands.

4. The system of claim 3 wherein said translation is communicated visually, audibly, or electronically.

5. The system of claim 2 further comprising one or more communication interface for communicating with a second human interface device.

6. The system of claim 2, wherein one or more of the human interface device, computer interface device, or a Morse Code decoder is contained in a headset.

7. The system of claim 2 further comprising one or more emergency devices for being actuated by said system to provide an emergency response actuating system comprising:

8. The system of claim 6 wherein said one or more emergency devices is an airbag, a parachute, a brake, an auto-pilot, or combinations thereof.

9. The system of claim 2 further comprising a storage medium for receiving and storing the received EEG signal, the decoded message, or combinations thereof, for providing a journaling or thought documenting system.

10. The system of claim 2 further comprising an adapter for converting the received EEG signal or Morse Code to one or more communication methods required for use of said electronic device, wherein said system allows for nonverbal and non-physical communication from human to a human interface device.

11. The system of claim 2 further comprising:
an input device for interfacing with said electronic device;
an encoder for translating the communication from said electronic device to Morse Code; and
one or more transmitters for transmitting said encoded communication to the one or more human interface devices;
wherein said system allows for nonverbal communication from an electronic device to a human interface device
